(19) United States
(12) Patent Application Publication
(10) Pub. No.: US 2017/0112789 A1
(43) Pub. Date: Apr. 27, 2017

(54) INTRAVENOUS BACLOFEN AND METHODS OF TREATMENT

(71) Applicants: Allaysis, LLC, Trevose, PA (US); Regents of the University of Minnesota, Minneapolis, MN (US)

(72) Inventors: Adolfo L. Gomez, Sicklerville, NJ (US); John Schrogie, Chester Springs, PA (US); Dan Simon Alhadeff, Weston, FL (US); Gilberto Diaz, Miramar, FL (US)

(21) Appl. No.: 15/331,757

(22) Filed: Oct. 21, 2016

Related U.S. Application Data
(60) Provisional application No. 62/244,537, filed on Oct. 21, 2015.

(51) Int. Cl. A61K 31/197; A61K 9/08; A61K 9/00

(52) U.S. Cl. CPC A61K 31/197; A61K 9/0019; A61K 9/08; A61K 9/0053

(57) ABSTRACT

An intravenous baclofen solution is disclosed, along with methods of dosing and treatment therewith.

### CROSS-REFERENCE TO RELATED APPLICATIONS

This application claims priority to U.S. provisional application No. 62/244,537 (the '537 application) entitled “INTRAVENOUS BACLOFEN AND METHODS OF TREATMENT,” filed 21 Oct. 2015. The '537 application is hereby incorporated by reference as though fully set forth herein.

### BACKGROUND OF THE INVENTION

Baclofen is a muscle relaxant and anti-spastic agent. Spasticity is a common symptom of upper motor neuron injury in individuals with cerebral palsy, multiple sclerosis, acquired spinal cord injury, brain injury, and neurodegenerative disorders. Baclofen is a structural analog of the inhibitory neurotransmitter gamma-aminobutyric acid (GABA) and acts as a GABA_{B} agonist at the level of the spinal cord. Baclofen is the generic name for 4-amino-3-(4-chlorophenyl) butanoic acid. It occurs naturally as a racemic mixture composed of R- and S-enantiomers. It is a white or off-white, mostly odorless crystalline powder with a molecular weight of 213.66, and it is slightly soluble in water. Baclofen's structural formula is

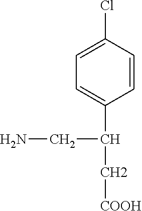 [SMILES: NCC(CC(=O)O)c1ccc(Cl)cc1]

The R-enantiomer and the S-enantiomer of baclofen are shown below.

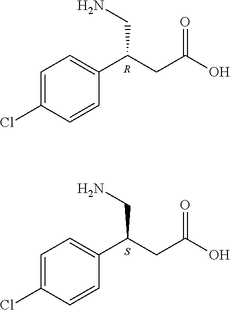

Racemic baclofen is sold under the tradename LIORESAL® in oral (10 mg or 20 mg tablets) and intrathecal (0.05 mg/mL, 0.5 mg/mL, or 2.0 mg/mL) formulations. In addition to being used to treat spasticity, oral baclofen has also been indicated for the treatment of other types of movement disorders, pain, addiction, depression and other psychological conditions, gastrointestinal disorders, respiratory disorders, sleep disorders, autism, and genetic disorders in either its racemic or R-enantiomer form. For example, U.S. Patent Application Publication No. 2010/0137442 discloses a sustained release oral dosage form of R-baclofen prodrugs and methods of treating multiple disease conditions, including neuropathic pain and muskuloskeletal pain. Nevertheless, it takes up to several hours for the mean plasma concentration of baclofen to reach therapuetic levels when the drug is administered orally.

Several circumstances exist in which patients treated with oral or intrathecal baclofen must abruptly discontinue therapy. For example, the intrathecal baclofen pump and/or catheter used in intrathecal administration may need to be removed, refilled, or replaced. Moreover, patients taking oral baclofen may be unable to do so during periods of illness, noncompliance, or surgery, for example. Abrupt discontinuation of oral or intrathecal baclofen can result in a severe withdrawal syndrome characterized by rebound increases in muscle tone and spasms, status epilepticus, hallucinations, and a neuromalignant syndrome-like picture potentially resulting in rhabdomyolysis and multisystem organ failure. The current recommended management of baclofen withdrawal is inadequate, and symptoms are often difficult to control. Agarwal et al., A Pilot Study Assessing Pharmacokinetics and Tolerability of Oral and Intravenous Baclofen in Healthy Adult Volunteers, J Child Neurol., (Jul. 14, 2014), http://jcn.sagepub.com/content/early/2014/07/11/0883073814535504.

There is a clinical need for a FDA-approved, commercially available intravenous balcofen formulation that can be used to quickly attain therapeutic plasma concentrations of baclofen for the initial treatment of acute or chronic pain, spasticity, and other conditions, as well as for the temporary treatment and prevention of withdrawal symptoms in patients whose oral baclofen therapy regimen has been interrupted.

### BRIEF SUMMARY OF THE INVENTION

An intravenous baclofen solution is disclosed, along with methods of dosing and treatment therewith. It is believed that intravenous administration of baclofen can permit rapid attainment of necessary drug concentrations, as well as accurate and precise dose titration. Thus, intravenous baclofen allows for more efficient and effective treatment of a variety of medical conditions, including baclofen withdrawal.

It should be noted that because baclofen is composed of R- and S-enantiomers, doses of the the R-enantiomer (R-baclofen) alone are 50% of those recommended when the racemic mixture is used. For purposes of this application (including the specification, claims, and drawings), listed amounts of baclofen refer to the racemic mixture unless otherwise specified.

One embodiment of the invention provides a method of treating a medical condition susceptible to baclofen therapy comprising: (a) administering to a patient a bolus intravenous dose of a therapeutically effective amount of a solution comprising baclofen at a concentration of up to about 2.0 mg/mL over a time period of about 5 minutes to about 60 minutes; (b) repeating administration of the bolus intravenous dose of baclofen until at least one of the following conditions is met: i) a total maximum dose of baclofen has been administered to the patient, ii) the patient experiences symptom control, or iii) the patient exhibits evidence of drug intolerance; (c) discontinuing administration of bolus intravenous doses of baclofen; and (d) administering to the patient an oral dose of a therapeutically effective amount of baclofen as needed for symptom control.

In another embodiment, a method of treating a medical condition susceptible to baclofen therapy, the method comprises: (a) administering to a patient an intravenous infusion of a therapeutically effective amount of a solution comprising baclofen at a concentration of about 2.0 mg/mL or less until at least one of the following conditions is met: i) a total maximum dose of baclofen has been administered to the patient, ii) the patient experiences symptom control, or iii) the patient exhibits evidence of drug intolerance; (b) discontinuing administration of the infusion of baclofen; and (c) administering to the patient an oral dose of a therapeutically effective amount of baclofen as needed for symptom control.

In another embodiment, a kit for treating a medical condition susceptible to baclofen therapy comprises: (a) a solution comprising baclofen at a concentration of up to about 2.0 mg/mL, the solution configured to be intravenously administered; (b) a plurality of baclofen tablets configured to be orally administered, each baclofen tablet comprising about 10-20 mg of baclofen; (c) instructions for i) intravenously administering a therapeutically effective amount of the solution to a patient during a first time period, and ii) orally administering a therapeutically effective amount of the plurality of baclofen tablets to the patient during a second time period.

Another embodiment of the invention provides a method of temporarily treating a patient with baclofen during a period of medical fluctuation that comprises (a) discontinuing oral or intrathecal administration of baclofen to the patient; (b) administering to the patient a bolus intravenous dose of a therapeutically effective amount of a solution comprising baclofen at a concentration of up to about 2.0 mg/mL over a time period of about 5 minutes to about 60 minutes; (c) repeating administration of the bolus intravenous dose of baclofen about every 6 to 8 hours until oral or intrathecal administration of baclofen can be resumed; (d) discontinuing administration of bolus intravenous doses of baclofen; and (e) resuming oral or intrathecal administration of baclofen.

In another embodiment, a method of intravenously administering baclofen to a patient that has previously been treated with oral baclofen in a therapeutically effective amount comprises (a) discontinuing oral administration of baclofen to the patient; (b) administering to the patient a bolus intravenous dose of solution comprising about 75% to about 85% of said amount of baclofen over a time period of about 5 minutes to about 60 minutes; (c) repeating administration of the bolus intravenous dose of baclofen about every 6 to 8 hours until oral administration of baclofen is resumed; (d) discontinuing administration of intravenous baclofen; and (e) resuming oral administration of baclofen.

In another embodiment, a method of temporarily treating a patient with baclofen during a period of medical fluctuation comprises (a) discontinuing oral or intrathecal administration of baclofen to the patient; (b) starting a continuous intravenous infusion of a therapeutically effective amount of a solution comprising baclofen at a concentration up to about 2.0 mg/mL over a time period of about 24 hours; (c) continuing the infusion about every 24 hours until oral or intrathecal administration of baclofen is resumed; (d) discontinuing the continuous intravenous infusion; and (e) resuming oral or intrathecal administration of baclofen.

In another embodiment, a method of intravenously administering baclofen to a patient that has previously been treated with oral baclofen in a therapeutically effective amount comprises (a) discontinuing oral administration of baclofen to the patient; (b) administering a continuous intravenous infusion of solution comprising about 75% to about 85% of said amount of baclofen over a time period of about 24 hours; (c) continuing the infusion about every 24 hours until oral administration of baclofen is resumed; (d) discontinuing administration of the continuous intravenous infusion; and (e) resuming oral administration of baclofen.

In another embodiment, a method of converting an oral dose of baclofen to an intravenous dose of baclofen comprises (a) determining the oral dose; and (b) multiplying the oral dose by between about 0.45 and about 1.0 to determine the intravenous dose.

In another embodiment, a pharmaceutical solution comprises an effective therapeutic amount of up to about 2.0 mg/mL baclofen dissolved in at least one of normal saline, dextrose solution, Lactated Ringer's solution, or any combination thereof; wherein the solution is adapted to be intravenously administered to a patient.

### BRIEF DESCRIPTION OF THE DRAWINGS

FIG. 1 is a graph of the mean (±standard deviation) plasma concentration-time profiles of racemic baclofen after intravenous and oral administration (0-12 hours).

FIG. 2 is a graph of the mean (±standard deviation) plasma concentration-time profiles of racemic baclofen after intravenous and oral administration at clinically relevant doses.

### DETAILED DESCRIPTION OF THE INVENTION

As used herein, the term “baclofen” refers to 4-amino-3-(4-chlorophenyl) butanoic acid or its pharmaceutically acceptable salt thereof or its derivatives. The term includes R-baclofen (the R enantiomer of baclofen), S-baclofen (the S enantiomer of baclofen), and the racemic mixture containing R-baclofen and S-baclofen in equal proportions. Amounts of baclofen listed in this application (including the specification, claims, and drawings) refer to the racemic mixture unless otherwise specified; equivalent amounts of R-baclofen can be extrapolated to be 50% of the racemic amount.

Each milliliter of intravenous baclofen solution can contain about 0.5 mg to about 2.0 mg of baclofen and an isotonic amount of sodium chloride dissolved in sterile water. In an embodiment, the concentration of baclofen in the intravenous solution can be about 0.5-2.0 mg/mL. In an embodiment, the intravenous baclofen solution can include a dextrose solution or Lactated Ringer's solution instead of or in combination with normal saline. The intravenous baclofen solution can further include an anticonvulsant drug, an antispasmodic drug, an anticholinergic drug, an antibiotic, a corticosteroid, an opioid, and/or a non-steroidal anti-inflammatory drug. The present invention also provides a correlation between oral and intravenous dosing of baclofen (see, e.g., Examples 2 and 3 below). In particular, an equivalent dose of intravenous baclofen can be determined by multiplying the oral dose of baclofen by about 0.45 to about 1.0, preferably by about 0.6 to about 0.9, and more preferably by about 0.75 to about 0.85.

Generally, an effective amount of the above-described intravenous baclofen solution can be administered intravenously to treat a patient presenting with acute or chronic moderate-to-severe pain. Both nociceptive pain (including somatic and visceral pain) and neuropathic pain can be treated with intravenous baclofen. For example, intravenous baclofen can be used to treat patients with lumbar pain, cancer pain, pain due to trauma, and trigeminal neuralgia.

In addition, an effective amount of the above-described intravenous baclofen solution can be administered intravenously to a patient presenting with other medical conditions that are susceptible to baclofen therapy. Examples of such medical conditions and indications for intravenous baclofen therapy include the following: spasticity and gastro-esophageal reflux disease (van Herwaarden et al., Aliment. Pharmacol. Ther. 2002, 16, 1655-62; Ciccaglione et al., Gut 2003, 52, 464-70; Andrews et al., U.S. Pat. No. 6,117,908; and Fara et al., International Publication No. WO 02/096404); alcohol abstinence in alcoholics (Gessa et al., International Publication No. WO 01/26638); smoking cessation (Gessa et al., International Publication No. WO 01/08675); reducing addiction liability of narcotic agents (Robson et al., U.S. Pat. No. 4,126,684); emesis (Bountra et al., U.S. Pat. No. 5,719,185); anti-tussive for the treatment of cough (Kreutner et al., U.S. Pat. No. 5,006,560); neuropathic pain such as trigeminal neuralgia (Bowsher, Br. Med. Bull. 1991, 47(3), 655-66; Fromm et al., Neurology 1981, 31, 683-7; and Ringel and Roy, Ann Neurol 1987, 21(5), 514-5), musculoskeletal pain such as low back pain (Dapas et al., Spine 1985, 10(4), 345-9; and Raphael et al., BMC Musculoskeletal Disorders 2002, 3(17), Epub 2002 Jun. 20); movement disorders such as dystonia and hiccups; peripheral nerve disorders such as muscle stimulation disorders; spinal cord disorders such as spastic paraparesis; other neurological disorders such as multiple sclerosis and cerebral palsy; sleep disorders such as narcolepsy (Black et al., Nuerobiol Dis 2014, 34(19), 6485-94); psychological conditions such as depression; as well as autism and fragile X syndrome (Welin, Age of Autism, Epub 2013 June 7, http://www.ageofautism.com/2013/06/roche-backs-out-on-seasie-therapeutics-drug-trial-for-fragile-x-parents-dismayed.html).

In addition, an effective amount of the above-described intravenous baclofen solution can be administered intravenously to temporarily treat a patient during a period of medical fluctuation resulting in the discontinuation of oral or intrathecal baclofen. Temporary treatment with intravenous baclofen may be necessary as bridging therapy (e.g., when a patient is temporarily unable to take oral or intrathecal baclofen) or for management of withdrawal symptoms, for example. As used herein, a “period of medical fluctuation” refers broadly to a time period during which a patient experiences an illness, condition, change in health status, or situation requiring an adjustment to his/her normal medical care or treatment plan. More specifically, a period of medical fluctuation can include at least one of a scheduled or unscheduled surgical procedure, trauma, ileus, bowel obstruction, vomiting, diarrhea, gastrointestinal malabsorption, seizure, stroke, subarachnoid hemorrhage, or patient non-compliance. Furthermore, for patients with implanted intrathecal pumps, periods of medical instability can include an intrathecal hardware failure or a necessity to remove, refill, or replace the intrathecal hardware.

In one embodiment of the invention, a method is provided to treat a medical condition susceptible to baclofen therapy. The method comprises administering to a patient a bolus intravenous dose of a therapeutically effective amount of a solution comprising baclofen (e.g., about 15 mg baclofen per bolus dose) at a concentration of up to about 2.0 mg/mL over a time period of about 5 minutes to about 60 minutes. Administration of the bolus intravenous dose of baclofen can be repeated until at least one of the following conditions is met: i) a total maximum dose of baclofen (e.g., about 60 mg) has been administered to the patient, ii) the patient experiences symptom control, or iii) the patient exhibits evidence of drug intolerance. Administration of the bolus intravenous dose of baclofen is then discontinued. Following discontinuation of the bolus intravenous dose, an oral dose of a therapeutically effective amount of baclofen (e.g., about 20 mg baclofen per oral dose) can be administered to the patient as needed for symptom control or relief. The patient can be medically evaluated, such as by a physician or other health care provider, within about seven days of beginning oral baclofen to determine whether the medication should be continued.

In another embodiment of the invention, a method of treating a medical condition susceptible to baclofen therapy comprises administering to a patient an intravenous infusion of a therapeutically effective amount of a solution comprising baclofen (e.g., about 15 mg baclofen per bolus dose) at a concentration of about 2.0 mg/mL or less until at least one of the following conditions is met: i) a total maximum dose of baclofen (e.g., about 60 mg) has been administered to the patient, ii) the patient experiences symptom control, or iii) the patient exhibits evidence of drug intolerance. Administration of the intravenous infusion of baclofen is then discontinued. Following discontinuation of the intravenous infusion, an oral dose of a therapeutically effective amount of baclofen (e.g., about 20 mg baclofen per oral dose) can be administered to the patient as needed for symptom control or relief. The patient can be medically evaluated, such as by a physician or other health care provider, within about seven days of beginning oral baclofen to determine whether the medication should be continued. It is believed that an intravenous infusion of baclofen can mimic intrathecal administration of baclofen, can eliminate or mitigate any peaks or troughs in baclofen levels in a patient's cerebral spinal fluid or blood (which may occur during oral or intravenous bolus administration, for example), and can reduce the risk or incidence of adverse events associated with intravenous baclofen.

In yet another embodiment of the invention, a kit is provided for treating a medical condition susceptible to baclofen therapy. The kit comprises a baclofen solution at a concentration of up to about 2.0 mg/mL for intravenous administration. The baclofen solution can be prepackaged in a bag or other container designed for intravenous administration. The kit can further comprise oral baclofen tablets (each tablet comprising about 10-20 mg baclofen), which can be prepackaged in a pillbox or similar container. Finally, the kit can also comprise instructions for intravenously administering (via bolus doses or infusion) a therapeutically effective amount of the solution to a patient during a first time period (e.g., up to about 24 hours) and for orally administering a therapeutically effective amount of oral baclofen tablets to the patient during a second time period (e.g., up to about 7 days). In an example, the contents of the kit can be packaged in a carrying case, suitable for use in a pocket or purse, or in a backpack.

In another embodiment of the invention, a method is provided to temporarily treat a patient with intravenous baclofen during a period of medical fluctuation that comprises discontinuation of oral or intrathecal administration of baclofen to the patient, followed by administration of an intravenous bolus dose of a therapeutically effective amount of a baclofen solution (e.g., between about 1 mg and about 50 mg baclofen per bolus dose) over a time period of about 5 to 60 minutes. The intravenous bolus dose can be administered repeatedly about every 6 to 8 hours until oral or intrathecal administration of baclofen can be resumed. Once intravenous bolus doing has been discontinued, oral or intrathecal administration of baclofen can be resumed.

In yet another embodiment of the invention, a method of temporarily treating a patient with intravenous baclofen during a period of medical fluctuation is provided that can include discontinuation of oral or intrathecal administration of baclofen, followed by administration of a continuous intravenous infusion of a therapeutically effective amount of a baclofen solution continued until oral or intrathecal administration of baclofen can be resumed. Once continuous intravenous infusions have been discontinued, oral or intrathecal administration of baclofen can be resumed. It is believed that continuous intravenous infusion of baclofen can mimic intrathecal administration of baclofen, can eliminate or mitigate any peaks or troughs in baclofen levels in a patient's cerebral spinal fluid or blood (which may occur during intravenous bolus administration, for example), and can reduce the risk or incidence of adverse events associated with intravenous baclofen.

The invention will be further described by the following detailed examples.

### EXAMPLE 1

### Preparation of Intravenous Racemic Baclofen Solution

108 g of sodium chloride were placed in a 600 ml beaker and then added to a 20 L beaker containing 8.4 L of sterile water for injection. The resulting sodium chloride solution was stirred in the 20 L beaker until dissolution occurred. Approximately 50 ml of sterile water for injection were used to rinse any residual sodium chloride from the 600 ml beaker, and the rinsings were added to the 20 L beaker. Next, 24.1 g of baclofen powder were placed in a 1 L beaker and then gradually (over a time period of about 2 hours) added and dissolved in the solution in the 20 L beaker. Approximately 50 ml of sterile water for injection was used to rinse any residual baclofen from the 1 L beaker, and the rinsings were added to the 20 L beaker. Approximately 3500 mL of sterile water for injection were then added to the 20 L beaker, and the solution was mixed for a minimum of 10 minutes to ensure homogeneity. The solution was then filtered using a Mini Kleenpak™ (0.2 μm) filter at 20 rpm.

An automated, aseptic filling machine was used to fill approximately 1090 13.5-mL glass vials with approximately 10.5 ml of the solution per vial. The vials were then stoppered, caps were placed on the vials, and the caps were crimped. The vials were stored at about 15 degrees Celsius to about 25 degrees Celsius.

### EXAMPLE 2

### Preparation of Intravenous R-Baclofen Solution (Prophetic Example)

54 g of sodium chloride are placed in a 300 ml beaker and then added to a 10 L beaker containing 4.2 L of sterile water for injection. The resulting sodium chloride solution is stirred in the 10 L beaker until dissolution occurs. Approximately 25 ml of sterile water for injection are used to rinse any residual sodium chloride from the 300 ml beaker, and the rinsings are added to the 10 L beaker. Next, 12 g of baclofen powder are placed in a 500 mL beaker and then gradually (over a time period of about 2 hours) added and dissolved in the solution in the 10 L beaker. Approximately 25 ml of sterile water for injection are used to rinse any residual baclofen from the 500 mL beaker, and the rinsings are added to the 10 L beaker. Approximately 1750 mL of sterile water for injection are then added to the 10 L beaker, and the solution is mixed for a minimum of 10 minutes to ensure homogeneity. The solution is then filtered using a Mini Kleenpak™ (0.2 μm) filter at 20 rpm.

An automated, aseptic filling machine is used to fill approximately 545 13.5-mL glass vials with approximately 5.25 ml of the solution per vial. The vials are then stoppered, caps are placed on the vials, and the caps are crimped. The vials are stored at about 15 degrees Celsius to about 25 degrees Celsius.

### EXAMPLE 3

### Administration of Intravenous Racemic Baclofen

A. Subjects

Twelve healthy human volunteers were recruited. The volunteers were medication free for 48 hours before, during, and 24 hours after the administration of the study drug.

B. Study Design

The 12 volunteers participated in a randomized, open-label, 2-way crossover study to compare the pharmacokinetics and bioavailability of oral baclofen with an intravenous baclofen formulation. The oral formulation was a 10 mg baclofen tablet. A single intravenous dose of 5 mg was administered over 15 minutes, using the commercially available 2 mg/mL intrathecal baclofen formulation (Lioresal Intrathecal). Blood samples (6 mL) for the measurement of plasma concentrations of baclofen were collected in blood collection tubes containing K2 ethylenediaminetetraacetic acid at the following times: prior to dosing; at 5, 15, and 30 minutes; and at 1, 2, 4, 6, 8, 10, 12, and 24 hours after drug administration.

C. Determination of Baclofen Concentration in Plasma

Study plasma samples were prepared by adding 50 μL of a 500 μg/mL levetiracetam solution (internal standard) to 250 μL of K₂ ethylenediaminetetraacetic acid (EDTA) human plasma. Baclofen and the internal standard were extracted from plasma by precipitating the protein with methanol and drying it under nitrogen at approximately 40° C. The dried residues were reconstituted in 300 μL of a mobile phase consisting of 20 mM ammonium acetate-methanol (75:25) solution. After 1 minute of vortex mixing, the reconstituted sample solution was filtered and injected onto the high-performance liquid chromatograph-mass spectrometer system. Standard curve samples over a range of 20 to 400 ng/mL baclofen and quality control samples containing 30 (low), 80 (medium), and 240 ng/mL (high) baclofen were prepared and analyzed in triplicate along with the study samples. The assay was linear over the range 20-400 ng/mL with a lower limit of quantification of 20 ng/mL.

Baclofen concentration-time data were analyzed using a noncompartmental pharmacokinetic approach with Phoenix software (version 6.2; Pharsight Corporation, Mountain View, Calif.). The terminal rate constant (λz) was determined from the slope of the terminal log-linear portion of the plasma concentration-time curve, and the terminal half-life (t_{1/2}) was calculated as In 2/(λz). Maximum plasma concentrations (Cₘₐₓ) and time to maximum concentration (Tₘₐₓ) were determined by direct observation of the data. The area under the concentration-time curve to the last nonzero plasma concentration (Cₗₐₛₜ) that was above half the lower limit of quantification (10 ng/mL) was calculated by the trapezoidal rule and reported as AUCₗₐₛₜ The area under the concentration-time curve extrapolated to infinity (AUC_{0-∞}) was calculated as AUCₗₐₛₜ+(Cₗₐₛₜ/λz). Mean and standard deviation values for the parameters were also obtained using the descriptive statistics tool in Phoenix version 6.2. A paired t-test was used to determine if statistical differences existed in log normalized, dose-adjusted area under the curve between oral and intravenous arms.

D. Results

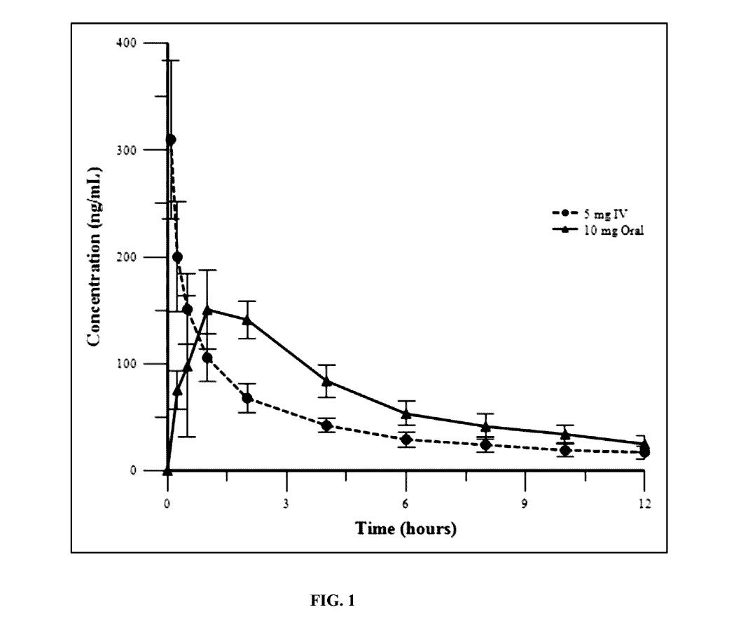

A summary of the pharmacokinetic parameters is presented in FIG. 1 and Table 1 below. The mean concentration-time profiles for both (5 mg intravenous and 10 mg oral) arms are shown in FIG. 1. When the subjects received the intravenous formulation, the observed maximum baclofen concentration occurred at the 5-minute time point, whereas the median Tmax for oral administration was 1 hour. The mean (standard deviation) Cmax values for the oral (10 mg) and intravenous (5 mg) doses were 174 (16) ng/mL and 310 (74) ng/mL, respectively (Table 1). The mean t_{1/2} was similar for both the oral and intravenous arms (4 and 4.52 hours, respectively). The mean absolute bioavailability of the oral baclofen tablets (Table 1) was 74%. There was a significant difference in log-normalized, dose-adjusted area under the curves (P=0.0024) between oral and intravenous dosing with similar variability (coefficient of variation: 18%-24%).

TABLE 1
Mean ± SD of Baclofen Pharmacokinetic Parameters
Following Oral (10 mg) and Intravenous (5 mg) Administration.

| | 5 mg IV | 10 mg oral |
|---|---|---|
| Pharmacokinetic parameter | (mean^{a} ± SD) | (mean ± SD) |
| Cₘₐₓ (ng/mL) | 310 ± 74 | 174 ± 16 |
| Tₘₐₓ (h)^{b} | — | 1.0 (0.5-2.0) |
| AUCₗₐₛₜ (ng · h/mL) | 593 ± 111 | 878 ± 199 |
| AUC_{0-∞} (ng · h/mL) | 707 ± 166 | 1023 ± 232 |
| AUC_{0-∞}/Dose^{c} (ng · h/mL/mg) | 141 ± 33 | 102 ± 23 |
| Bioavailability (%) | — | 74 ± 15 |
| T_{1/2} (h) | 4.52 ± 1.6 | 4.03 ± 0.73 |

Abbreviations:
AUC, area under the curve;
IV, intravenous;
SD, standard deviation.
^{a}Median (min, max) are presented as arithmetic means
^{b}Median (min, max) reported for Tₘₐₓ.
^{c}Two-tailed P value < .05 (paired t-test performed on dose-normalized area under the curve)

The intravenous formulation was well tolerated. All treatment-emergent adverse events were characterized by the investigator as being mild in severity, and all subjects returned to their baseline values within 6 hours of drug administration.

E. Discussion

This example demonstrates that intravenous administration of baclofen results in therapeutic mean plasma concentrations of the drug much more quickly than does oral administration of baclofen. As noted above, the maximum plasma concentration of baclofen was reached in five minutes with intravenous administration versus one hour with oral administration. Therefore, intravenous administration of baclofen may be preferred over oral administration when a patient presents with newly diagnosed pain, spasticity, or other conditions susceptible to baclofen therapy. Initial treatment with intravenous baclofen can provide quicker symptom control than treatment with oral baclofen. Alternatively, temporary treatment with intravenous baclofen during an interruption of chronic oral or intrathecal therapy can prevent or mitigate symptoms of baclofen withdrawal.

This example also illustrates that absolute oral baclofen bioavailability is about 75%, indicating that approximately 25% of a 10-mg dose is either not absorbed or undergoes first-pass metabolism prior to drug reaching systemic circulation. This suggests that a smaller intravenous baclofen dose can be used when it is substituted for an oral dose. For example, assuming linear kinetics, the total systemic exposure (area under the curve) after an intravenous dose of 15 mg would be equivalent to the total exposure achieved after 20 mg of oral baclofen dose. Thus, this example suggest that when intravenous baclofen is substituted for oral baclofen, an equivalent intravenous dose will be about 75% of the oral dose.

In one embodiment of the invention, a method of converting an oral dose of baclofen to an intravenous dose of baclofen comprises (a) determining the oral dose; and (b) multiplying the oral dose by between about 0.45 and about 1.0 to determine the intravenous dose. In another embodiment of the invention, a method of converting an oral dose of baclofen to an intravenous dose of baclofen comprises (a) determining the oral dose; and (b) multiplying the oral dose by between about 0.6 and about 0.9 to determine the intravenous dose. In yet another embodiment of the invention, a method of converting an oral dose of baclofen to an intravenous dose of baclofen comprises (a) determining the oral dose; and (b) multiplying the oral dose by about 0.75 to determine the intravenous dose.

### EXAMPLE 4

### Intravenous Racemic Baclofen Dose Escalation

A. Subjects

Thirty-six healthy adults participated in the study.

B. Study Design

The thirty-six subjects were enrolled in a randomized, open-label, cross over study to evaluate the safety profile and pharmacokinetics of oral and an investigational intravenous baclofen formulation at clinically relevant doses. Three cohorts of twelve subjects received single doses of baclofen: 7.5, 11.5 or 15 mg intravenously and 10, 15, or 20 mg taken orally with a 48 hour washout phase. The highest dosing cohort received an additional 15 mg extended (60 minute) infusion. Blood samples for baclofen pharmacokinetic analysis were collected pre-dose and at regular intervals over a 24 hour time period. Over this 24 hour period subjects were assessed for sedation, nystagmus and ataxia to evaluate clinical response. Maximum concentration (Cₘₐₓ) and time to maximum concentration (Tₘₐₓ) were determined by visual inspection. Noncompartmental pharmacokinetic analyses were used to estimate area under the curve (AUC), half life (t_{1/2}), clearance (CL) and volume of distribution. Absolute bioavailability was calculated using dose normalized oral and intravenous AUCs. Dose proportionality was assessed using analysis of variance (ANOVA) and a power model. Bioequivalence was evaluated using Phoenix Software (Centara, Pharsight Corporation, Mountain View, Calif.).

C. Results

1. Tolerability/Safety

Mild, self-limited adverse effects were seen following both oral and intravenous baclofen doses. Adverse events associated with known pharmacological effects of baclofen included sedation, ataxia, and nystagmus. With regard to sedation, changes from baseline were not statistically significant either by route of administration or by dose. Regarding ataxia, all subjects could walk without assistance, and one subject experienced mild transient ataxia after the 20 mg oral dose. Of the twelve subjects in each dosing arm: two, six, and six subjects experienced asymptomatic nystagmus following 10 minute infusions of 7.5 mg, 11.5 mg, and 15 mg, respectively.

2. Pharmacokinetics

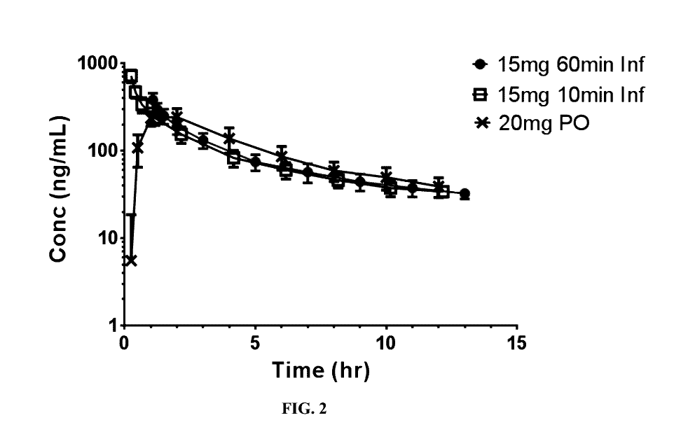

A summary of the pharmacokinetic parameters is presented in FIG. 2 and Table 2 below. The mean plasma concentration-time profiles for baclofen following a 20 mg oral dose, a 15 mg intravenous infusion over 10 minutes, and a 15 mg intravenous infusion over 60 minutes are shown in FIG. 2. The mean Cₘₐₓ following intravenous administration was 2.5 to 2.8 times higher than following oral doses; however, the area under the curves (exposure) were similar following oral and intravenous administration. The mean Cₘₐₓ increased proportionally with dose for both forms of administration, and the AUC displayed a greater than proportional increase with dose based on ANOVA and power model analysis. The mean t_{1/2} was similar for both oral and intravenous baclofen administration, and it appeared to increase with dose. The oral and intravenous doses were bioequivalent based on AUC, but not based on Cₘₐₓ. Extending the 15 mg intravenous infusion from 10 to 60 minutes, however, decreased the Cₘₐₓ ration from 263% to 149%.

TABLE 2
Mean (+/−SD) Baclofen Pharmacokinetic Parameters Following oral and
intravenous administration

| Pharmacokinetic | PO | | | 10 min Infusion | | | 60 min Infusion |
|---|---|---|---|---|---|---|---|
| Parameters | 10 mg | 15 mg | 20 mg | 7.5 mg | 11.5 mg | 15 mg | 15 mg |
| Cₘₐₓ | 124 ± | 201 ± | 255 ± | 310 ± | 524 ± | 720 ± | 382 ± |
| (ng/mL) | 30.9 | 57.6 | 53.7 | 88.0 | 150 | 119 | 73.9 |
| AUG_{(0-inf)} | 714 ± | 1201 ± | 1598 ± | 562 ± | 1186 ± | 1520 ± | 1447 ± |
| (ng · hr/mL) | 157 | 242 | 367 | 134 | 286 | 244 | 276 |
| Bioavailability | 102 ± | 80 ± | 85 ± | — | — | — | — |
| (%) | 15 | 18 | 18 | | | | |
| T_{1/2} | 3.94 ± | 4.65 ± | 5.49 ± | 3.37 ± | 4.97 ± | 5.82 ± | 6.22 ± |
| (hr) | 1.76 | 1.43 | 0.88 | 1.52 | 1.60 | 1.13 | 1.53 |

D. Discussion and Conclusions

All oral and IV doses were generally well tolerated although some subjects experienced sedation and mild nystagmus. Occurrence of nystagmus appears to increase with increasing IV doses given as 10 minute infusions; however, this was not observed following the 15 mg 60 minute infusion. This suggests that nystagmus may be related to Cₘₐₓ. The absolute oral bioavailability ranged from 42% to 132% with a mean bioavailability of 89% (±19) when data from all cohorts were combined. However, because there were limited baclofen concentrations above the limit of quantification for cohort 1, the bioavailability estimates for cohort 2 and 3 may be more reliable. When the bioavailability estimates for cohort 2 and 3 are considered, an IV dose 15-20% smaller than an oral dose would be needed to produce equivalent total systemic exposure. Increases in baclofen Cₘₐₓ was proportionate to the increase in baclofen dose based on a power model and ANOVA. Thus, it is feasible to use intravenous baclofen to attain therapeutic plasma concentrations of baclofen for the initial treatment of acute or chronic pain, spasticity, and other conditions, as well as to temporarily treat and prevent withdrawal symptoms in patients whose oral baclofen therapy regimen has been interrupted.

Although several embodiments have been described above with a certain degree of particularity, those skilled in the art could make numerous alterations to the disclosed embodiments without departing from the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

Any patent, publication, or other disclosure material cited above is incorporated by reference herein as though fully set forth. Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. A method of treating a medical condition susceptible to baclofen therapy, the method comprising:
(a) administering to a patient a bolus intravenous dose of a therapeutically effective amount of a solution comprising baclofen at a concentration of up to about 2.0 mg/mL over a time period of about 5 minutes to about 60 minutes;
(b) repeating administration of the bolus intravenous dose of baclofen until at least one of the following conditions is met: i) a total maximum dose of baclofen has been administered to the patient, ii) the patient experiences symptom control, or iii) the patient exhibits evidence of drug intolerance;
(c) discontinuing administration of bolus intravenous doses of baclofen; and
(d) administering to the patient an oral dose of a therapeutically effective amount of baclofen as needed for symptom control.

2. The method of claim 1, wherein the solution comprises baclofen at a concentration of about 0.5-2.0 mg/mL.

3. The method of claim 1, wherein the solution comprises baclofen at a concentration of about 0.5-1.0 mg/mL.

4. The method of claim 1, wherein symptom control comprises relief from pain or spasticity.

5. The method of claim 1, wherein the bolus intravenous dose comprises up to about 15 mg of baclofen.

6. The method of claim 1, wherein the oral dose comprises about 20 mg of baclofen.

7. The method of claim 1, further comprising medically evaluating the patient within about 7 days of performing step (d).

8. The method of claim 1, wherein the administering step (a) occurs over a time period of about 10 minutes.

9. The method of claim 1, wherein the total maximum dose of baclofen is about 60 mg.

10. The method of claim 1, wherein the medical condition comprises at least one of spasticity and other movement disorders, pain, addiction, depression and other psychological conditions, gastrointestinal disorders, respiratory disorders, sleep disorders, autism, and genetic disorders.

11. The method of claim 1, wherein the baclofen comprises at least one of R-baclofen or a racemic mixture baclofen.

12. The method of claim 11, wherein the baclofen comprises R-baclofen, and wherein the solution comprises R-baclofen at a concentration of us to about 1.0 mg/mL.

13. A method of treating a medical condition susceptible to baclofen therapy, the method comprising:
(a) administering to a patient an intravenous infusion of a therapeutically effective amount of a solution comprising baclofen at a concentration of about 2.0 mg/mL or less until at least one of the following conditions is met: i) a total maximum dose of baclofen has been administered to the patient, ii) the patient experiences symptom control, or iii) the patient exhibits evidence of drug intolerance;
(b) discontinuing administration of the infusion of baclofen; and
(c) administering to the patient an oral dose of a therapeutically effective amount of baclofen as needed for symptom control.

14. The method of claim 13, wherein the solution comprises baclofen at a concentration of about 0.5-2.0 mg/mL.

15. The method of claim 13, wherein the solution comprises baclofen at a concentration of about 0.5-1.0 mg/mL.

16. The method of claim 13, wherein symptom control comprises relief from pain or spasticity.

17. The method of claim 13, wherein the continuous intravenous infusion comprises up to about 15 mg of baclofen per hour.

18. The method of claim 13, wherein the oral dose comprises about 20 mg of baclofen.

19. The method of claim 13, further comprising medically evaluating the patient within about 7 days of performing step (c).

20. The method of claim 13, wherein the total maximum dose of baclofen is about 60 mg.

21. The method of claim 13, wherein the medical condition comprises at least one of spasticity and other movement disorders, pain, addiction, depression and other psychological conditions, gastrointestinal disorders, respiratory disorders, sleep disorders, autism, and genetic disorders.

22. The method of claim 13, wherein the baclofen comprises at least one of R-baclofen or a racemic mixture baclofen.

23. The method of claim 22, wherein the baclofen comprises R-baclofen, and wherein the solution comprises R-baclofen at a concentration of us to about 1.0 mg/mL.

24. A kit for treating a medical condition susceptible to baclofen therapy, the kit comprising:
(a) a solution comprising baclofen at a concentration of up to about 2.0 mg/mL, the solution configured to be intravenously administered;
(b) a plurality of baclofen tablets configured to be orally administered, each baclofen tablet comprising about 10-20 mg of baclofen;
(c) instructions for i) intravenously administering a therapeutically effective amount of the solution to a patient during a first time period, and ii) orally administering a therapeutically effective amount of the plurality of baclofen tablets to the patient during a second time period.

25. The kit of claim 24, wherein the solution comprises baclofen at a concentration of about 0.5-2.0 mg/mL.

26. The kit of claim 24, wherein the solution comprises baclofen at a concentration of about 0.5-1.0 mg/mL.

27. The kit of claim 24, wherein the first time period comprises up to about 24 hours.

28. The kit of claim 24, wherein the second time period comprises up to about 7 days.

29. The kit of claim 24, wherein the solution is not intravenously administered to the patient during the second time period.

30. The kit of claim 24, wherein the plurality of baclofen tablets is not orally administered to the patient during the first time period.

31. The kit of claim 24, wherein the solution is configured to be intravenously administered in bolus doses or as an infusion.

32. The method of claim 24, wherein the medical condition comprises at least one of spasticity and other movement disorders, pain, addiction, depression and other psychological conditions, gastrointestinal disorders, respiratory disorders, sleep disorders, autism, and genetic disorders.

33. The method of claim 24, wherein the baclofen comprises at least one of R-baclofen or a racemic mixture baclofen.

34. The method of claim 33, wherein the baclofen comprises R-baclofen, and wherein the solution comprises R-baclofen at a concentration of up to about 1.0 mg/mL.

35. A method of temporarily treating a patient having a medical condition susceptible to baclofen therapy with baclofen during a period of medical fluctuation, the method comprising:
(a) discontinuing oral or intrathecal administration of baclofen to the patient;
(b) administering to the patient a bolus intravenous dose of a therapeutically effective amount of a solution comprising baclofen at a concentration of up to about 2.0 mg/mL over a time period of about 5 minutes to about 60 minutes;
(c) repeating administration of the bolus intravenous dose of baclofen about every 6 to 8 hours until oral or intrathecal administration of baclofen is resumed;
(d) discontinuing administration of bolus intravenous doses of baclofen; and
(e) resuming oral or intrathecal administration of baclofen.

36. The method of claim 35, wherein the medical condition comprises at least one of spasticity and other movement disorders, pain, addiction, depression and other psychological conditions, gastrointestinal disorders, respiratory disorders, sleep disorders, autism, and genetic disorders.

37. The method of claim 35, wherein the baclofen comprises at least one of R-baclofen or a racemic mixture baclofen.

38. The method of claim 35, wherein the baclofen comprises R-baclofen, and wherein the solution comprises R-baclofen at a concentration of up to about 1.0 mg/mL.

39. The method of claim 35, wherein the medical fluctuation comprises at least one of the following: an intrathecal hardware failure, a necessity to remove, refill, or replace intrathecal hardware, a scheduled or unscheduled surgical procedure, trauma, ileus, bowel obstruction, vomiting, diarrhea, gastrointestinal malabsorption, seizure, stroke, subarachnoid hemorrhage, or patient non-compliance.

40. The method of claim 35, wherein the solution comprises baclofen at a concentration of about 0.5-2.0 mg/mL.

41. The method of claim 35, wherein the solution comprises baclofen at a concentration of about 0.5-1.0 mg/mL.

42. The method of claim 35, wherein the patient is experiencing baclofen withdrawal symptoms.

43. A method of intravenously administering baclofen to a patient having a medical condition subject to baclofen therapy and presently being treated with oral baclofen in a therapeutically effective amount, the method comprising:
(a) discontinuing oral administration of baclofen to the patient;
(b) administering to the patient a bolus intravenous dose of solution comprising about 75% to about 85% of said amount of baclofen over a time period of about 5 minutes to about 60 minutes;
(c) repeating administration of the bolus intravenous dose of baclofen about every 6 to 8 hours until oral administration of baclofen is resumed;
(d) discontinuing administration of intravenous baclofen; and
(e) resuming oral administration of baclofen.

44. The method of claim 43, wherein the medical condition comprises at least one of spasticity and other movement disorders, pain, addiction, depression and other psychological conditions, gastrointestinal disorders, respiratory disorders, sleep disorders, autism, and genetic disorders.

45. The method of claim 43, wherein the baclofen comprises at least one of R-baclofen or a racemic mixture baclofen.

46. The method of claim 45, wherein the baclofen comprises R-baclofen, and wherein the solution comprises R-baclofen at a concentration of up to about 1.0 mg/mL.

47. The method of claim 43, further comprising performing steps (a)-(d) during a period of medical fluctuation; wherein the medical fluctuation comprises at least one of the following: an intrathecal hardware failure, a necessity to remove, refill, or replace intrathecal hardware, a scheduled or unscheduled surgical procedure, trauma, ileus, bowel obstruction, vomiting, diarrhea, gastrointestinal malabsorption, seizure, stroke, subarachnoid hemorrhage, or patient non-compliance.

48. The method of claim 43, wherein the solution comprises baclofen at a concentration of about 0.5-2.0 mg/mL.

49. The method of claim 43, wherein the solution comprises baclofen at a concentration of about 0.5-1.0 mg/mL.

50. A method of temporarily treating a patient having a medical condition patient to baclofen therapy with baclofen during a period of medical fluctuation, the method comprising:
(a) discontinuing oral or intrathecal administration of baclofen to the patient;
(b) starting a continuous intravenous infusion of a therapeutically effective amount of a solution comprising baclofen at a concentration of about 2.0 mg/mL or less over a time period of about 24 hours;
(c) continuing the infusion until oral or intrathecal administration of baclofen is resumed;
(d) discontinuing the continuous intravenous infusion; and
(e) resuming oral or intrathecal administration of baclofen.

51. The method of claim 50, wherein the medical condition comprises at least one of spasticity and other movement disorders, pain, addiction, depression and other psychological conditions, gastrointestinal disorders, respiratory disorders, sleep disorders, autism, and genetic disorders.

52. The method of claim 50, wherein the baclofen comprises at least one of R-baclofen or a racemic mixture baclofen.

53. The method of claim 52, wherein the baclofen comprises R-baclofen, and wherein the solution comprises R-baclofen at a concentration of up to about 1.0 mg/mL.

54. The method of claim 50, wherein the medical fluctuation comprises at least one of the following: an intrathecal hardware failure, a necessity to remove, refill, or replace intrathecal hardware, a scheduled or unscheduled surgical procedure, trauma, ileus, bowel obstruction, vomiting, diarrhea, gastrointestinal malabsorption, seizure, stroke, subarachnoid hemorrhage, or patient non-compliance.

55. The method of claim 50, wherein the solution comprises baclofen at a concentration of about 0.5-2.0 mg/mL.

56. The method of claim 50, wherein the solution comprises baclofen at a concentration of about 0.5-1.0 mg/mL.

57. The method of claim 50, wherein the patient is experiencing baclofen withdrawal symptoms.

58. A method of intravenously administering baclofen to a patient having a medical condition susceptible to baclofen therapy and presently being treated with oral baclofen in a therapeutically effective amount, the method comprising:
(a) discontinuing oral administration of baclofen to the patient;
(b) administering to the patient a continuous intravenous infusion of solution comprising about 75% to about 85% of said amount of baclofen over a time period of about 24 hours;
(c) repeating administration of the continuous intravenous infusion about every 24 hours until oral administration of baclofen is resumed;
(d) discontinuing the continuous intravenous infusion; and
(e) resuming oral administration of baclofen.

59. The method of claim 58, wherein the medical condition comprises at least one of spasticity and other movement disorders, pain, addiction, depression and other psychological conditions, gastrointestinal disorders, respiratory disorders, sleep disorders, autism, and genetic disorders.

60. The method of claim 58, wherein the baclofen comprises at least one of R-baclofen or a racemic mixture baclofen.

61. The method of claim 60, wherein the baclofen comprises R-baclofen, and wherein the solution comprises R-baclofen at a concentration of us to about 1.0 mg/mL.

62. The method of claim 58, further comprising performing steps (a)-(d) during a period of medical fluctuation; wherein the medical fluctuation comprises at least one of the following: an intrathecal hardware failure, a necessity to remove, refill, or replace intrathecal hardware, a scheduled or unscheduled surgical procedure, trauma, ileus, bowel obstruction, vomiting, diarrhea, gastrointestinal malabsorption, seizure, stroke, subarachnoid hemorrhage, or patient non-compliance.

63. The method of claim 58, wherein the solution comprises baclofen at a concentration of about 0.5-2.0 mg/mL.

64. The method of claim 58, wherein the solution comprises baclofen at a concentration of about 0.5-1.0 mg/mL.

65. A method of converting an oral dose of baclofen to an intravenous dose of baclofen, the method comprising:
(a) determining the oral dose; and
(b) multiplying the oral dose by between about 0.45 and about 1.0 to determine the intravenous dose.

66. The method of claim 65, wherein the baclofen comprises at least one of R-baclofen or a racemic mixture baclofen.

67. The method of claim 65, wherein step (b) comprises multiplying the oral dose by between about 0.6 and about 0.9 to determine the intravenous dose.

68. The method of claim 65, wherein step (b) comprises multiplying the oral dose by between about 0.7 and about 0.8 to determine the intravenous dose.

69. A pharmaceutical solution comprising
an effective therapeutic amount of up to about 2.0 mg/mL baclofen dissolved in at least one of normal saline, dextrose solution, Lactated Ringer's solution, or any combination thereof;
wherein the solution is adapted to be intravenously administered to a patient.

70. The solution of claim 69, wherein the baclofen comprises at least one of R-baclofen or a racemic mixture of baclofen.

71. The solution of claim 69, wherein the baclofen comprises R-baclofen, and wherein the solution comprises R-baclofen at a concentration of up to about 1.0 mg/mL.

72. The solution of claim 69, wherein the patient has a medical condition comprising at least one of spasticity and other movement disorders, pain, addiction, depression and other psychological conditions, gastrointestinal disorders, respiratory disorders, sleep disorders, autism, and genetic disorders.

73. The solution of claim 69, further comprising at least one of an anticonvulsant drug, an antispasmodic drug, an anticholinergic drug, or an antibiotic drug.

74. The solution of claim 69, wherein the solution is sterile.

75. The solution of claim 69, wherein the solution comprises baclofen at a concentration of about 0.5-2.0 mg/mL.

76. The solution of claim 69, wherein the solution comprises baclofen at a concentration of about 0.5-1.0 mg/mL.
